(19) United States
(12) Patent Application Publication
(10) Pub. No.: US 2008/0214896 A1
(43) Pub. Date: Sep. 4, 2008

(54) ENDOSCOPE WITH DETACHABLE ELONGATION PORTION

(76) Inventors: Robert J. Krupa, Leominster, MA (US); Thomas V. Root, Beverly, MA (US); William F. Laflash, Northbridge, MA (US); Anthony J. Parillo, Sherborn, MA (US); Aleksandra A. Levshina, Leominster, MA (US)

Correspondence Address: PROSKAUER ROSE LLP, ONE INTERNATIONAL PLACE, BOSTON, MA, 02110, US

(21) Appl. No.: 11/972,489

(22) Filed: Jan. 10, 2008

Related U.S. Application Data
(60) Provisional application No. 60/879,718, filed on Jan. 10, 2007.

(51) Int. Cl. A61B 1/008; A61B 1/00

(52) U.S. Cl. 600/136; 600/141

(57) ABSTRACT

Endoscopes comprising a handle and a detachable elongation portion housing a light source for illuminating the distal end of the elongation portion are provided.

### RELATED APPLICATIONS

This application claims the benefit of U.S. Provisional Patent Application No. 60/879,718, filed on Jan. 10, 2007, the entire teachings of which are incorporated herein by reference.

### BACKGROUND

Endoscopes are devices used for viewing a region of an object that has limited access (e.g., inside a human being or animal, inside a pipe, inside an engine). Typically, an endoscope has a manipulation portion or handle coupled to an elongated portion so that, during use of the endoscope, the manipulation portion remains outside of the object while the elongated portion is at least partially disposed therein. In general, the elongated portion has one or more optical components to illuminate and view the region inside the object, and the manipulation portion has one or more devices designed to control the optical components and position the elongated portion within the object.

To provide suitable light to the elongation portion, so that proper visual images can be obtained, conventional endoscopes use external light sources that are tethered to the endoscope. The connection between the light source and the optical channel extending through the endoscope needs to be properly aligned so that light can be efficiently transmitted through the scope. If the light source and the optical channel are misaligned, not enough light will be transmitted through the channel and an operator will be unable to use the endoscope for visualization. As a result, it is difficult to exchange the elongation portion of an endoscope for a different sized elongation portion or a clean elongation portion, because doing so requires that the light source be disconnected from the old elongation portion and reconnected and aligned to the new elongation portion. In addition to aligning the light source an operator will also have to properly align the optics within the endoscope so that clear images can be transmitted therethrough.

Therefore, a need remains for an endoscope which will allow an operator to easily disconnect and attach a new and/or different elongation portion to the manipulation portion.

### SUMMARY OF THE TECHNOLOGY

In one aspect, the technology features a detachable elongation portion for an endoscope. The elongation portion has a detachment section, a shaft, and a light guide. In some embodiments, the detachment section includes a first light source, and is configured such that the elongation portion can be removably attached to an endoscope handle. The shaft has a distal end and a proximal end. The proximal end of the light guide is coupled to the first light source and the light guide extends through the shaft from the proximal end to the distal end.

In another aspect, the technology features an endoscope comprising a detachable elongation portion and a handle. In some embodiments, the elongation portion comprises a detachment section comprising a first light source, the detachment section configured such that the detachable elongation portion can be removably attached to an endoscope handle, a shaft comprising a distal end and a proximal end, a light guide wherein a proximal end of the light guide is coupled to the first light source and wherein the light guide extends through the shaft to the distal end of the shaft.

In some embodiments, endoscopes including a handle and an elongation portion are provided. The handle includes an attachment portion and the elongation portion includes a distal end and a proximal end. The distal end is adapted for visualizing an object and the proximal end houses a light source for illumination the object. The proximal end of the elongation portion is secured to the attachment portion of the handle.

Endoscope handles are provided. The endoscope handle can comprise a power source and a first attachment interface for removably connecting the handle to an elongation portion. The first attachment interface can comprise a contoured surface configured to match a contour of a second attachment interface of the elongation portion and a connection member for removably connecting the power source to a light source in the elongation portion.

Elongation portions for an endoscope are also provided. The elongation portion can comprise a shaft, a detachment section, and a light guide. The shaft comprises a distal end and a proximal end. The detachable section comprises at least one light source and a first attachment interface. The first attachment interface can comprise a contoured surface configured to match a contour of a second attachment interface of an endoscope handle and a connection member for removably connecting the light source to a power source in the handle.

Endoscopes comprising a handle and an elongation portion capable of being removably coupled to the handle are provided. The endoscope handle can comprise a power source and a first attachment interface. The elongation portion can comprise a shaft, a detachment section, and a light guide. The shaft comprises a distal end and a proximal end. The detachable section comprises a light source and a second attachment interface. The endoscope can also comprise a connecting device for removably connecting the power source to the light source, and an attachment device for securing the handle to the elongation portion, wherein the handle comprises a first member of the attachment device and the elongation portion comprises a second member of the attachment device. The first attachment interface comprises a contoured surface configured to match a contoured surface of the second attachment interface.

In some embodiments of the handle and/or endoscopes provided herein, the handle comprises a power source, a joystick coupled to a gear system, and a first attachment interface comprising at least a portion of the gear system. The elongation portion comprises a shaft, a detachable section, and a light guide. The shaft comprises a distal end and a proximal end. The detachable section includes a light source and at least one wire coupled to the distal end of the shaft and to one or more gears present in the detachable section. The light guide extends from the distal end of the shaft into the detachable section where it is coupled to the light source. The detachable section also comprises a second attachment interface comprising a portion of the one or more gears. The handle can also include a connecting device for removably connecting the power source to the light source, and an attachment device for securing the handle to the elongation portion, wherein the handle comprises a first member of the attachment device and the elongation portion comprises a second member of the attachment device. The first and second attachment interfaces are configured to allow the removable coupling of the elongation portion to the handle such that the gear system of the handle meshes with the one or more gears of the detachment section to form a mechanism for articulating the distal end of the elongation portion.

Methods of connecting a handle to an elongation portion of an endoscope to provide visualization of an object are also provided. In some embodiments, the method comprises providing a handle, the handle including an attachment interface and a control device for operating a light source. Providing an elongation portion including a shaft comprising a distal end for visualization of the object, a proximal end that is joined to a detachment section, the detachment section housing a light source and being securable to the attachment interface of the handle; and attaching the detachable section of the elongation portion to the attachment interface of the handle so that the elongation portion is secured to the handle and so that the control device for operating the light source is operable.

Methods of replacing an elongation portion of an endoscope are also provided. In some embodiments, the method comprises providing an endoscope as described herein; detaching the proximal end of the elongation potion from the attachment surface of the handle; providing a replacement elongation portion, and securing the detachment section of the replacement elongation portion to the attachment portion of the handle.

The technology permits the detachment of the elongation portion of the endoscope from the handle. As a result, an operator can detach the elongation portion without the use of specialized equipment. Moreover, the operator can insert and/or attach a new, different, or repaired elongation portion to the handle such that light source and the light guide will properly align such that light is efficiently transmitted through the endoscope. The elongation portion of an endoscope can be exchanged with another elongation portion without the need for realigning the light guide and/or the light source. The technology gives an operator the flexibility to change the elongation portion whenever he or she wishes (e.g., to replace a broken part, to attach a cleaned elongation portion, to attach an elongation portion with a different size lens, length or diameter). The technology also maximizes the battery life of an energy source for a light source through the use of a power controller, such as a pulse width modulating circuit. As a result, battery life can be extended while still providing a stable intensity light source. The technology also stores digital images on a digital mass storage device. As a result, images captured by the camera can be reviewed at a later date for further analysis.

The various embodiments described herein can be complimentary and can be combined or used together in a manner understood by the skilled person in view of the teachings contained herein.

### BRIEF DESCRIPTION OF THE DRAWINGS

FIG. 1 is an exemplary endoscope showing a cross-sectional view of a detachable elongation portion and a handle, where the elongation portion is attached to the handle.

FIG. 2 is an exemplary endoscope showing a detachable elongation portion attached to a handle, with a cross-sectional view of the elongation portion revealing light sources, light guides, wires, and at least one gear.

FIG. 3 is an exemplary light pattern that an endoscope can project onto an object for calculating properties relevant to an inspection.

FIG. 4 is an exemplary endoscope showing a detachable elongation portion attached to a handle, with the cross-sectional views of the elongation portion and the handle revealing light sources, light guides, wires, and gears.

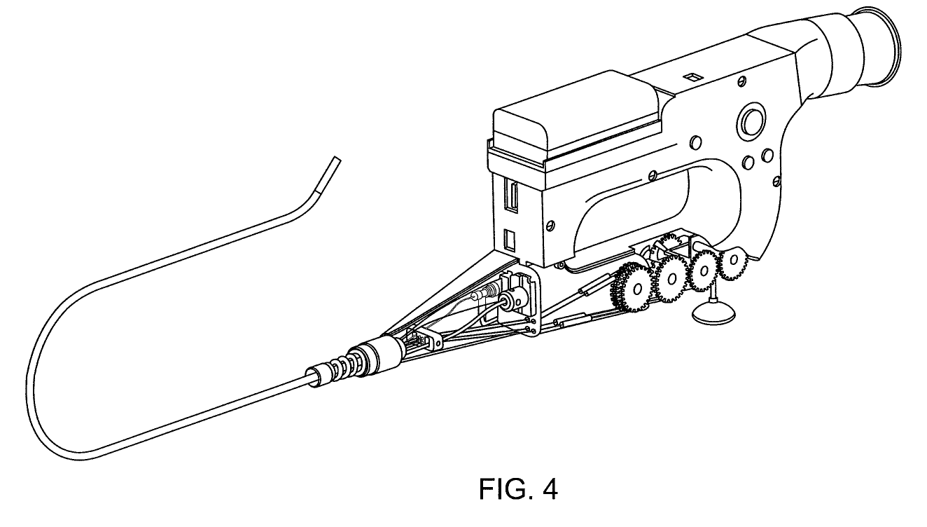

FIG. 5 is an exemplary endoscope illustrating the removable attachment of the elongation portion to the handle.

### DETAILED DESCRIPTION OF THE TECHNOLOGY

Provided herein are handheld endoscopes with a handle and a detachable elongation portion (hereinafter also referred to as “insertion tube,” “insertion section,” or “insertion shaft”). The detachable elongation portion can house a light source (also referred to herein as a “first light source”) for illuminating an object. In some embodiments, the detachable elongation portion of the endoscope also houses a second light source for calculating properties (e.g. measurements, distances, sizes) with respect to the inspected object and the elongation portion.

In some embodiments, the detachable elongation portion includes a light source, a light guide, a connection member or attachment section for connecting the light source to the power source (also referred to herein as “energy source”), and a member of an attachment device (also referred to herein as “securing latch”) for securing the elongation portion to the handle. Where the elongation portion includes an articulatable distal end, the elongation portion can include articulation wires and one or more gears.

In some embodiments, the handle includes a power source, and a member of an attachment device for securing an elongation portion to the handle, and optics for viewing the object of interest at the distal end of the elongation portion. In this embodiment, the optical viewing is accomplished with a camera and associated optics at the distal end of the detachable shaft. Power and signals to and from the camera are transmitted through a connector in the insertion shaft that mates to a connector on the handle.

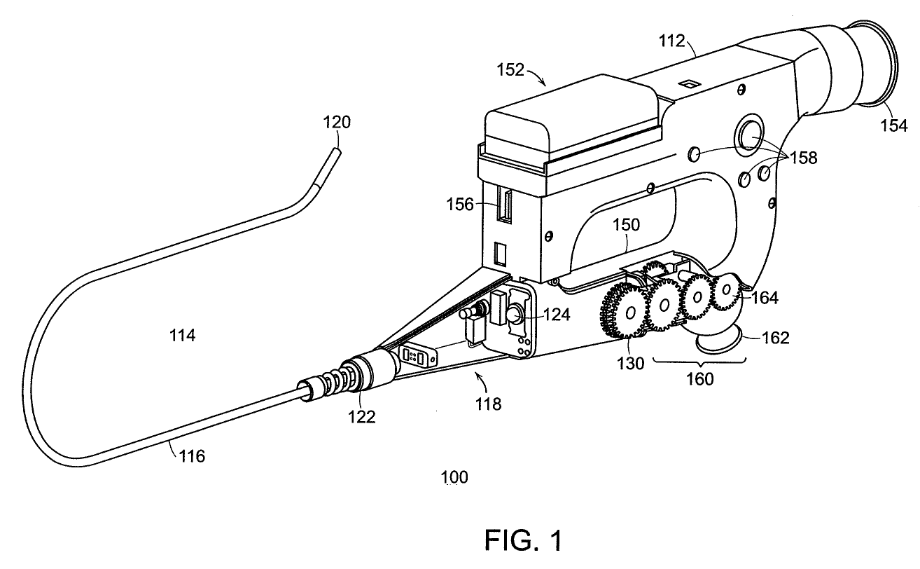

Referring to FIG. 1, in some embodiments, the endoscope 100 includes a detachable elongation portion 114 and a handle 112. The detachable elongation portion 114 includes a distal end 120, a shaft 116, a proximal end 122, a detachment section 118, and a gear 130. The handle 112 includes an attachment portion 150, a power source 152, an eyepiece 154, an optional USB port 156, controls for light and power 158, and in some embodiments, an articulation mechanism 160. The articulation mechanism 160 comprises a control lever 162 and a gear or set of gears 164 coupled to the control lever 162. In some embodiments, the eyepiece is a video display, which displays the image collected by the camera in the distal end 120 of the insertion shaft 114. The gear or gears 164 are configured such that when the detachable elongation portion 114 and the handle 112 are attached to each other, the gear 130 of the elongation portion 114 meshes with one or more gears 164 of the handle 112 thereby forming a articulation mechanism whereby operation of the control lever 162 articulates the distal end 120 of the elongation portion in the desired direction.

The control lever 162 herein provided by the endoscope can be any mechanism for articulating the distal end 120 of the elongation portion 114. For example, the control lever 162 can be a joystick. In some embodiments, the control lever 162 can control a two- or four-way articulation mechanism as described herein. In some embodiments, the control lever 162 can control an all-way articulation mechanism. For example, the control lever 162 can be a joystick controller such as the joystick controller disclosed in U.S. Pub. No. 2004/0059191 to Krupa et al.

The endoscopes provided herein can include an optional display for displaying images captured by the camera or other visualization device in the distal end 120 of the shaft 116, thus enabling the operator to view readily the object under inspection. The display can be on the handle 112 or external to the handle 112. The display can be a micro display, such as a microVGA (Kopin). The display can be activated when the endoscope is ready for use, for example, when the elongation portion 114 is connected to the handle 112. Disconnecting the elongation portion 114 from the handle 112 can deactivate the display, thereby enhancing the lifetime of the power source 152.

The endoscopes provided herein can include a slot for an optional digital storage device. The digital storage device stores images transmitted by the camera or other visualization device in the distal end 120 of the shaft 116. The digital storage device can store images or video. For example, the digital storage device can be a thumb drive, digital card, data card, and/or flash card. The images, either standalone or within a video clip, can be saved in formats such as TIFF, JPEG, MPEP, and/or GIFF files. The digital storage device can be removed for playback on an external device, and it can be left in the endoscope 100 for playback on the optional display.

The endoscopes provided herein can include an optional image capturing mechanism. When the operator engages the image capturing mechanism, a single image from the camera or visualization device in the distal end 120 of the shaft 116 can be captured. Alternatively, engaging the image capturing mechanism can begin the capture of a stream of images for a video. The image capturing mechanism can be, for example, a freeze frame button, and the image or video can be the images being displayed on the optional display. The image capturing mechanism can be located on the handle 112. The endoscope 100 can store a data imprint with every captured image, and the imprint can include information such as the date, time, inspector, part number, item inspected, measurement, and pointers to indicate flaw area of interest or any area of interest.

The endoscopes provided herein can optionally transmit data wirelessly to an external device. The data can be images and/or video, and the data can be stored either before or after transmission. Alternatively, the endoscope 100 can transmit data to an external device through an S-Video connection. The external device can be a remote monitor, a monitor headset, a laptop, and/or a desktop computer.

The remote monitor can be directly connected via S-Video connection or by wireless connection, thus allowing the remote monitor to be untethered as well. The endoscope may also be connected to a laptop with a video capture board. A laptop based upon the Citadel field hardened laptop design may be provided as part of the system.

The endoscopes provided herein can include additional controls or indicators on the handle 112. The controls can include, for example, an optional power setting for a light source and/or an optional button for calculating properties with respect to the inspected object and the elongation portion. The indicators can include, for example, a battery level indicator and/or an indication of the elongation portion insertion onto the engine (safe/unsafe).

The endoscopes provided herein can include one or more protective mechanisms for protecting the controls or other components on the handle 112. For example, the protective mechanism can be a covering, such as a sealed membrane switch keypad.

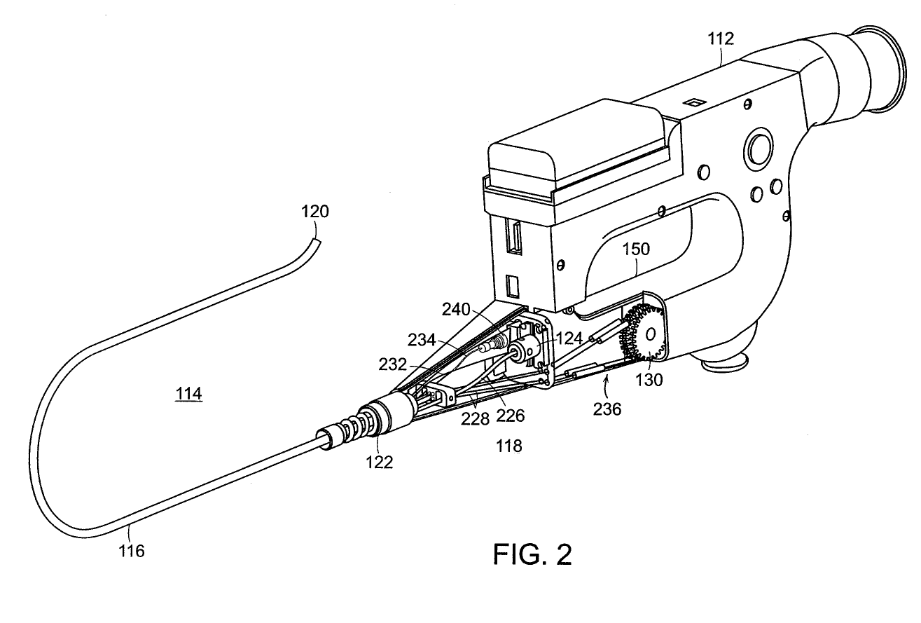

Referring to FIG. 2, in some embodiments, the endoscope includes a detachable elongation portion 114 and a handle 112. The elongation portion 114 includes a shaft portion 116 and a detachment section 118. The shaft portion 116 comprises distal end 120 and a proximal end 122. A light source 124 is contained within the detachment section 118. The light source 124 is positioned such that light generated by the light source 124 is able to be transmitted into the light guide 226 which runs from the light source 124 through the shaft 116 to the distal end 120. The distal end 120 can include a lens or other visualization device such as a camera or video camera, so that light transmitted through the elongation portion 114 can be used to illuminate a nearby object (e.g., an object located with 1 mm to 1000 mm from the distal end) and/or so that the camera can obtain a visual image of the object.

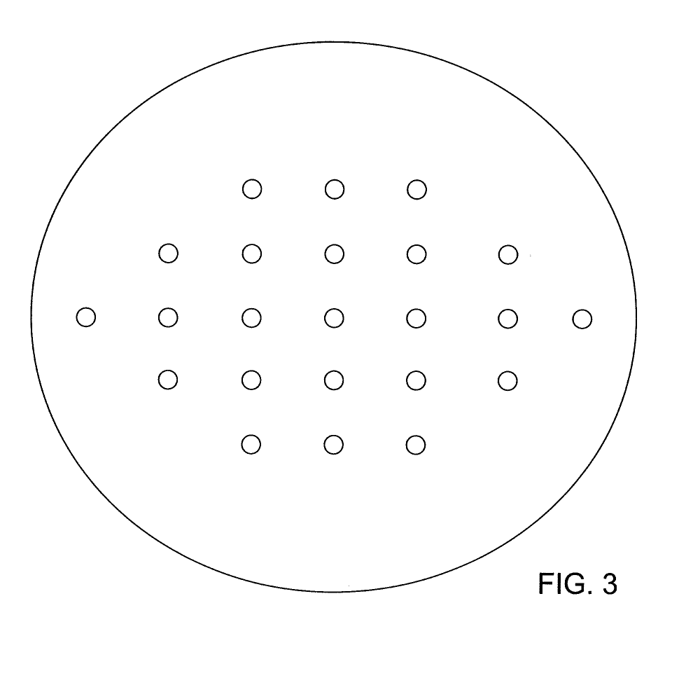

The endoscopes provided herein can include an optional second light source 240. The second light source 240 can be, for example, a laser, a semiconductor light source, a semiconductor diode laser, or a solid state light source. The light source 240 is coupled to a second light guide 234 running from the proximal end 122 to the distal end 120. At the distal end 120, the light guide 234 can have an opaque covering with a pattern, as shown in FIG. 3. Light transmitted through the light guide 234 from the light source 240 projects an illuminated pattern upon the object. The pattern can be used to calculate properties relevant to the inspection such as the size of the illuminated object, the distance between the distal end 120 and the object, and/or the tilt between the distal end and the object.

The optional laser source is used to illuminate an optional laser fiber 234 that extends to the distal end of the shaft where an optional pattern generator is illuminated. A laser light pattern is thus transmitted to the object being viewed and can be used to determine, for example, the size of the object.

In one embodiment, the detachable elongation portion comprises an attachment portion 236. The attachment portion 236 comprises a contoured surface that is configured to match a contoured portion of an attachment surface 150 of the handle. Where the elongation portion comprises an articulating distal end, the attachment portion 236 of the elongation portion includes one or more gears 130. When the elongation portion is attached to the handle, the gear or gears 130 mesh with a gear or gears present in the handle (see FIG. 1).

In the embodiment shown in FIG. 2, the shaft 116 comprises a flexible material and an articulating distal end that is capable of four way articulation. Two sets of articulation wires 228 are shown. The articulation wires are connected to the distal end 120 of the shaft and to gears 130 in the detachment section of the elongation portion.

The endoscopes provided herein can include an optional power source that can deliver power to a display on the handle 112. The elongation portion 114 of the endoscope can include electrical wires 232 that can connect to the power source when the elongation portion 114 is connected to the handle 112. When the elongation portion 114 and the handle 112 are connected, power can flow from the power source through the electrical wires 232 to an optional camera or video camera present in the distal end of the shaft. The power source can also be used to supply power to the first light source and to an optional laser source.

In some embodiments, the power source in the handle 112 is a battery such as a rechargeable lithium ion battery. The power source can contain enough energy to power the light source 124 continuously for at least 2-4 hours. An operator can remove the power source from the handle 112 once the power source is depleted and insert a new or recharged power source. Alternatively, the operator can connect the endoscope to an external AC or DC power source.

The light source can be any suitable light source for transmitting light from one end of a light guide to another. The light source can be coupled to the light guide by any suitable means, such as a lens system. In some embodiments, the light source 124 can be, for example, a semiconductor light source, a solid state light source, a light emitting diode, a semiconductor diode, or a semiconductor diode laser. The light source 124 can be the light source disclosed in U.S. Pub. No. 2004/0246744 by Krupa et al. The light source 124 can be a light emitting diode (LED) such as the Luxeon III Model LXHL-LW3C. The light source 124 can have a high color temperature, a high intensity, an emitting surface of approximately 1 mm×1 mm, and/or a coating with a wavelength conversion phosphor and/or fluorophore that emits a broadband continuum of visible light. The light source 124 can have a spectral output such as ultraviolet, 430 nm, 470 nm, 530 nm, near infrared, or infrared. The light source 124 can be a flat surface light source. The light source 124 can be round or cylindrical. Furthermore, the light source 124 can have a small surface mount package. The light source 124 can be a light emitting diode enclosed in an epoxy dome lens, where the epoxy dome is removed to the level of the light emitting diode. The light source can comprise the light emitting surface with an optional wavelength conversion phosphor in the absence of any encasement or gel, such as an index matching gel, such that the light guide can be coupled to the light source such that the light emitting surface or the wavelength conversion phosphor contacts the proximal end of the light guide.

In some embodiments of the elongation portion, the shaft 116 can have a diameter of any size, for example, 4 mm, 6 mm, or 8 mm. The shaft 116 can have a length of any dimension. Furthermore, the shaft 116 can exhibit any level of rigidity. For example, the shaft 116 can be made of flexible, rigid, or semi-rigid material. The shaft 116 can comprise a design and/or material that provide a smooth surface from the handle all the way to the distal tip, such as a continuous braid of tungsten. The braid design can be the design disclosed in, for example, U.S. Pat. No. 6,991,603 by Krupa et al. The shaft 116 can comprise a design that enhances flexibility, such as a vertebrae design. The shaft 116 can comprise a material that does not work harden with flexure, such as tungsten. Alternatively, the shaft 116 can be a catheter-based shaft. The endoscopes provided herein can include an optional shaft stiffener. The shaft stiffener can be placed on the distal end of an elongation portion to make the distal end behave like a rigid scope.

The distal tip can be attached using a technique that prevents the edge of the distal tip from being exposed. Such a technique reduces or eliminates the likelihood of catching the end tip upon scope retraction.

The endoscopes provided herein can include a distal end 120 capable of two- or four-way articulation. Alternatively, the distal end 120 can lack articulation, altogether.

In some embodiments, the elongation portion can include an articulatable portion at the distal end. The articulatable portion can be controlled by the operator so that the distal end can be articulated. As a result, the operator can move the distal end to a position close to and/or in a proper orientation for viewing a particular object. The articulatable portion is controlled through the use of strings or wires extending from the articulatable portion through the elongation portion to an articulation mechanism. FIG. 2 shows an elongation portion that includes a four way articulating distal end. However, in other embodiments, the shaft can be rigid, or can be flexible but without an articulating distal end, or can include a two-way, instead of a four-way articulating distal end. A two-way articulating distal end can comprise one set of wires connected to the distal end and to a gear within the detachment section of the elongation portion.

The elongation portions provided herein can include a detachment section that comprises a mating connector. The handle provided herein can include a connector that connects with the mating connector of the elongation portion when the handle is connected to the elongation portion.

The endoscopes provided herein can include a measurement system, such as a laser projection system. The projection system provides measurements for more accurate multi-point triangulation, without the need for surface contour modeling. Alternatively, the endoscope can include a scope proximity sensor. The laser projection system can comprise, for example, a semiconductor light source, such as a diode laser, coupled into a light guide, such as a fiber optic light guide. This light guide transmits the laser light down the length of the shaft. At the distal end of the shaft, the laser light exits the light guide and illuminates a target. This target, in this embodiment, is a thin film of metal deposited onto the back side of a GRIN (gradient index lens) into which several openings are present. Where these openings occur, laser light passes through the target (or mask) and is imaged by the GRIN lens onto the object being inspected. This pattern of dots, FIG. 3, is then used to determine the size and distance of an object.

The technology allows an operator to use interchangeable elongation portions of different parameters, such as diameter and length, with the same endoscope handle. Each elongation portion has the same detachment section, thus enabling any elongation portion to be used with a single handle. However, as the different parameters affect the processes of capturing images and calculating properties, whenever an elongation portion is attached to an endoscope handle, the handle must be calibrated to account for the properties of the elongation portion. For example, the electronics in the handle can store one or more look-up calibration tables corresponding to different types of elongation portions, where the values in the tables depend upon properties of the elongation portions, such as diameter and length. When an elongation portion is attached to the handle, the electronics in the handle retrieved the appropriate calibration look-up table based upon the attached elongation portion.

For example, the elongation portion can store a code, such as a binary code, that indicates its type. The type can correspond to properties of the elongation portion such as diameter, length, or any other property, or any combination thereof. The code can be stored, for example, in one or more pins on the elongation portion that plug into receptacles on the handle. The receptacles can be connected to the electronics in the handle. Thus, when an operator attaches an elongation portion to a handle, the code stored on the pins can connect to receptacles on the handle, and the receptacles can transmit the information stored on the pins to the electronics in the handle. Based on the transmitted information, the appropriate calibration look-up table is selected.

The endoscope provided herein can include an optional power controller connected to the power source. The power controller can be a pulse width modulating circuit. The power controller can enable the light source to emit an approximately constant intensity of light by promoting a stable power draw from the power source. In some embodiments, a power controller, such as a pulse width modulating circuit, is provided in combination with the energy source. The power controller promotes a stable energy draw off of the energy source so that the light source can emit light having a substantially constant intensity. In general, the shorter the pulse width of the modulating circuit, the longer the energy source will maintain enough energy to power the light source.

The endoscope provided herein can include an optional control device that enables the user to turn the light source 124 on or off. The control device further allows the user to control the intensity of the light source 124.

The endoscope provided herein can allow the visualization devices in the distal end of the elongation portion to be removed and replaced with upgraded devices having compatible diameters.

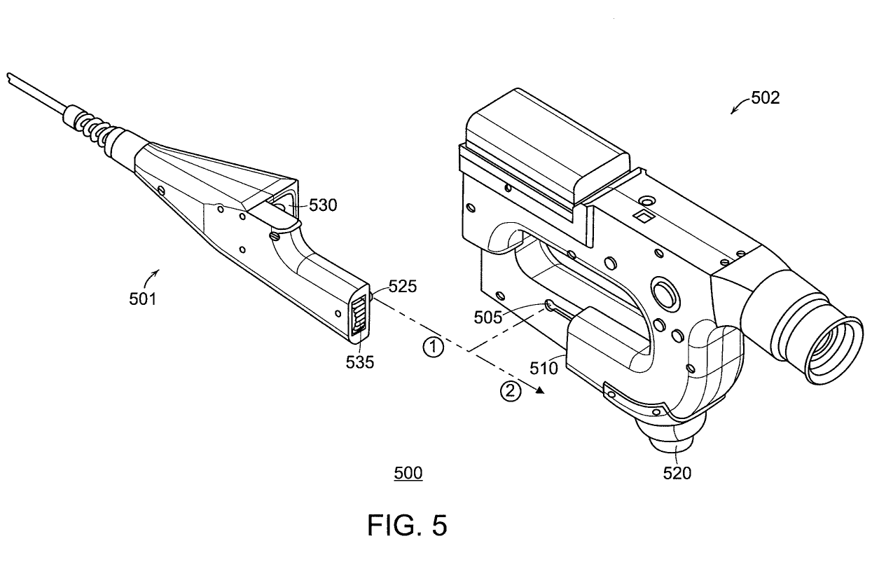

FIG. 5 is an exemplary endoscope 500 with an elongation portion 501 and a handle 502. In some embodiments, the handle 502 has a keyway 505, a connector 510, gears 515, an articulation mechanism 520, and a securing latch. The elongation portion 501 has a shaft key 525, a mating connector 530, and gears 535. To attach the elongation portion 501 to the handle 502, the shaft key 525 is inserted into the keyway 505. The elongation portion 501 is pushed towards the handle 502, aligning the connector 510 at the front of the handle with the mating connector 530 on the elongation portion. The gears between the elongation portion and the handle 535, 515 are aligned by lighting moving the articulation mechanism 520 until the gears set. Finally, the securing latch is closed between the handle 502 and the elongation portion 501.

To detach the elongation portion 501 from the handle 502, the articulation mechanism 520 is moved to its home position. The securing latch is unhooked between the elongation portion 501 and the handle 502. The shaft key 525 is pulled back to unhook the elongation portion 501 from the handle 502. The elongation portion 501 is slowly pulled straight out from the front of the handle 502 until the shaft key 525 is at the end of the keyway 505. Finally, the shaft key 525 is pulled out of the keyway 505.

In some embodiments, to disconnect the elongation portion including the articulatable portion from the handle, the operator first decouples the proximal end from the handle by, for example, untwisting the twist lock provided at the proximal end of the elongation portion. The operator then removes the elongation portion by pulling it away from the handle while disengaging the wire drum in the elongation portion from the gear system in the handle.

To connect a new or replacement elongation portion to the handle, the operator first slides the attachment portion of the elongation portion into the attachment portion of the handle such that the wire drum of the elongation portion meshes with the gear system of the handle and such that the light source is connected to the power source. Next the operator secures the elongation portion to the handle by activating an attachment device, such as a twist lock.

In some embodiments, the securing latch can be a twist lock or a bayonet mount detachment system.

In some embodiments, the endoscope is fully sealed and the insertion tube is immersible in water, fuels, and other common liquids. In some embodiments, there is a ground point that will allow for a ground attachment for those instances where grounding is desired.

In some embodiments, the endoscope can be used as a stand alone unit without wires connecting the endoscope to external power, display or computer equipment. In some embodiments, the endoscope includes wireless transmission capability to transmit data, images, and or video to an external computer.

Because of the placement of the light source within the detachment section of the elongation portion, the technology provided herein allows for the exchange of detachable elongation portions attached to an endoscope handle without the need to reconnect and realign the light source. Thus, the operator can avoid misalignment that can render the light source ineffective. As the elongation portion can be attached or detached from the handle without disturbing the connection between the light source and the light guide, the maximum amount of light can be transmitted from the light source to the distal end of the elongation portion.

The technology provided herein allows for insertion shaft components that are not integrated with the handle components. The elongation portions and handles provided herein are able to “stand alone” and once attached, function as a cohesive unit. As a result, an operator can use the same handle for different inspections requiring elongation portions with different diameters and/or lengths. For example, if the elongation portion attached to the handle is inadequate for an inspection, the operator can detach the elongation portion and attach an elongation portion whose diameter and/or length are more suitable. In addition, when an endoscope becomes damaged or otherwise unusable, the operator can return for repair the damaged or unusable insertion shaft instead of the entire endoscope. For example, the operator can detach the unusable elongation portion from the handle, attach a functioning elongation portion, send the unusable elongation portion to the appropriate place for repair, and continue conducting inspections while the unusable elongation portion is being repaired. In addition, the operator can replace the elongation portion in the field without the use of special tools or equipment.

The technology provided herein allows the endoscope to be used in the absence of an umbilical cord (the endoscope can be untethered). Without an external light source and the necessary means to connect the external light source to a power source, the endoscope becomes easier to transport, set up, and use. Furthermore, the light source can be easily cooled since air or any other cooling medium can access the proximal end of the elongation portion.

The endoscopes, handles, and elongation portions provided herein may be embodied in other specific forms without departing from the spirit or essential characteristics thereof. The foregoing embodiments are therefore to be considered in all respects illustrative rather than limiting on the endoscopes, handles, and elongation portions described herein.

What is claimed is:

1. A detachable elongation portion for an endoscope comprising:
a detachment section comprising a first light source, the detachment section configured such that the detachable elongation portion can be removably attached to an endoscope handle,
a shaft comprising a distal end and a proximal end, and
a light guide, wherein a proximal end of the light guide is coupled to the first light source and wherein the light guide extends through the shaft to the distal end of the shaft.

2. The detachable elongation portion of claim 1, wherein the proximal end of the light guide is coupled to the first light source such that the proximal end of the light guide is in contact with a light emitting surface of the first light source.

3. The detachable elongation portion of claim 1, wherein the proximal end of the light guide is coupled to the first light source such that the proximal end of the light guide is in contact with a wavelength converting substance.

4. The detachable elongation portion of claim 1, wherein the first light source is a semiconductor light source.

5. The detachable elongation portion of claim 1, further comprising a second light source located in the detachment section.

6. The detachable elongation portion of claim 5, wherein the second light source is part of a system for calculating properties related to the elongation portion and the object under inspection.

7. The detachable elongation portion of claim 6, wherein the second light source is a semiconductor light source.

8. The detachable elongation portion of claim 6, wherein the second light source is coupled to a proximal end of a second light guide.

9. The detachable elongation portion of claim 6, wherein the second light guide extends through the shaft to the distal end of the shaft.

10. The detachable elongation portion of claim 1, further comprising a parameter setting device including a code that sets parameters in electronics in the endoscope handle when the elongation portion is attached to the endoscope handle.

11. The detachable elongation portion of claim 1, wherein the first light source further comprises an attachment section configured to connect to a power source in an endoscope handle when the elongation portion is connected to the handle.

12. The detachable elongation portion of claim 1, further comprising a mechanical articulating device for articulating the distal end of the shaft.

13. The detachable elongation portion of claim 14, wherein the mechanical articulating device comprises gears that mesh with gears on an endoscope handle.

14. An endoscope comprising a detachable elongation portion and a handle wherein the elongation portion comprises a detachment section comprising a first light source, the detachment section configured such that the detachable elongation portion can be removably attached to an endoscope handle, a shaft comprising a distal end and a proximal end, a light guide wherein a proximal end of the light guide is coupled to the first light source and wherein the light guide extends through the shaft to the distal end of the shaft.

15. The endoscope of claim 14, wherein the first light source is a semiconductor light source.

16. The endoscope of claim 14, further comprising a second light source located in the detachment section.

17. The endoscope of claim 16 wherein the second light source is part of a system for calculating properties related to the elongation portion and the object under inspection.

18. The endoscope of claim 16, wherein the second light source is a semiconductor light source.

19. The endoscope of claim 16, wherein the second light source is coupled to a proximal end of a second light guide.

20. The endoscope of claim 14, wherein the second light guide extends through the shaft to the distal end of the shaft.

21. The endoscope of claim 16, wherein the handle further comprises a receiving device and the elongation portion further comprises a parameter setting device, the receiving device being configured to connect with the parameter setting device.

22. The endoscope of claim 21, wherein the parameter setting device comprises a code whereupon connection with the receiving device sets parameters in electronics of the handle based upon the code.

23. The endoscope of claim 14, wherein the first light source further comprises an attachment section configured to connect to a power source located in an endoscope handle when the elongation portion is connected to the handle.

24. The endoscope of claim 14, wherein the detachable elongation portion further comprises a mechanical articulating device for articulating the distal end of the shaft.

25. The endoscope of claim 14, wherein the detachable elongation portion further comprises a first gear system and the handle further comprises a second gear system, wherein the first gear system is configured to mesh with the second gear system to form a system for controlling the articulating device of the elongation portion.
